(19) United States
(12) Patent Application Publication
(10) Pub. No.: US 2026/0083574 A1
(43) Pub. Date: Mar. 26, 2026

(54) FREE HAND NAVIGATION INSTRUMENTS FOR TOTAL HIP ARTHROPLASTY

(71) Applicant: GLOBUS MEDICAL, INC., AUDUBON, PA (US)

(72) Inventors: Israel Oluwasakin, Dresher, PA (US); Hayden Cameron, Philadelphia, PA (US)

(21) Appl. No.: 18/897,137

(22) Filed: Sep. 26, 2024

(51) Int. Cl. A61F 2/46; A61B 17/17; A61B 34/20

(52) U.S. Cl. CPC A61F 2/4609; A61B 17/1703; A61B 17/1746; A61B 34/20; A61B 2034/2051; A61F 2002/4632

(57) ABSTRACT

Methods provide computer-navigation assisted total hip arthroplasty (THA) procedures. Such procedures include the use of a navigated instrument, e.g., a navigated reamer construct or a navigated inserter construct to prepare the acetabulum and insert an acetabular shell, a camera tracking system adapted to track a pose of the navigated inserter or reamer construct relative to the patient in use, and a computer platform adapted to receive pose tracking information and generate and display navigational guidance for the user.

### BACKGROUND

The disclosure relates generally to devices, systems, and methods for use in computer navigation-assisted surgical procedures. More particularly, the disclosure relates to methods, systems, and devices for performing computer navigation-assisted bone preparation and implant placement processes during total hip arthroplasty (THA) procedures.

Hip arthroplasty, or hip replacement, is a surgical procedure used to resurface and reconstruct a hip joint that has been damaged by disease or injury, e.g., by arthritis or a fracture. THA devices replace both the acetabulum and the femoral head that collectively comprise the hip joint. An acetabular implant (or “shell”) is secured to the acetabulum, which may be prepared by reaming the acetabular surface prior to implant placement. Once fixated in the acetabulum, the implant forms a replacement articulating surface which interfaces with the femoral implant secured to the end of the femur. The femoral implant is pivotably coupled to the acetabular implant, thereby reconstructing the hip joint. Exemplary acetabular implants are disclosed in, e.g., U.S. patent application Ser. No. 17/024,876, filed Sep. 18, 2020 (published as US 2022/0087823 A1), which is incorporated by reference as though fully set forth herein.

Surgeons typically develop preoperative plans which are useful to determine appropriate angles and positions for implant placement during surgery. Manual guides and/or intra-operative fluoroscopy may be relied upon to determine appropriate positioning of instruments and/or implants during surgery. Although current surgical approaches offer sophisticated techniques for obtaining accurate information regarding instrument and implant positions during surgery in order to prepare the bones and accurately place implants according to the pre-operative plan, current approaches may have shortcomings, e.g., in THA procedures. Improvements in acetabular component placement processes are desirable to reduce operating time while attaining proper component positioning, thereby reducing the burden of negative surgical outcomes such as impingement, dislocation, pelvic osteolysis, wear, and need for early revision.

### BRIEF DESCRIPTION OF THE DISCLOSURE

A first aspect of the disclosure provides a method for preparing an acetabulum of a patient in a total hip arthroplasty (THA) procedure, comprising: assembling a navigated reamer construct including a reamer basket; providing a camera tracking system adapted to track a pose of the navigated reamer construct relative to the patient, wherein the camera tracking system is in communication with a computer platform; inserting the reamer basket of the navigated reamer construct into the acetabulum; displaying navigation guidance to a user, wherein the navigation guidance comprises one or more of a depth and a planned center of an acetabular shell; reaming the acetabulum with the navigated reamer construct based on the navigation guidance displayed to the user, to remove damaged bone therefrom; and removing the navigated reamer construct from acetabulum.

In certain embodiments, assembling the navigated reamer construct comprises operatively coupling a power system to a reamer driver; affixing the reamer basket of a selected size to a distal end of the reamer driver; and inputting data indicating the selected size of the reamer basket into the computer platform. In certain additional embodiments, assembling the navigated reamer construct comprises affixing a tracking array to the reamer driver, wherein the tracking array comprises a plurality of reference elements adapted for tracking by the camera tracking system.

In certain embodiments, inputting the data indicating the selected size of the reamer basket further comprises using a touch screen or an extended reality (XR) interaction with an XR headset.

In certain embodiments, displaying the navigation guidance includes displaying the navigation guidance to the user on one or more of a display screen or an extended reality (XR) headset in communication with the computer platform.

In certain embodiments, the method includes performing two or more iterations of any two or more processes in the method selected from: the assembling, inserting, displaying, reaming, and removing processes. In further additional embodiments, a first selected size of the reamer basket is used in a first iteration, and a second selected size of the reamer basket is used in a second iteration.

In certain embodiments, a patient reference array is affixed to a rigid anatomical feature of the patient prior to the inserting, wherein the patient reference array comprises a plurality of reference elements adapted for tracking by the camera tracking system.

A second aspect of the disclosure provides a method for placing an acetabular shell in a prepared acetabulum of a patient in a total hip arthroplasty (THA) procedure, comprising assembling a navigated inserter construct; providing a camera tracking system adapted to track a pose of the navigated inserter construct relative to the patient, wherein the camera tracking system is in communication with a computer platform; displaying navigation guidance to a user; inserting the acetabular shell into the prepared acetabulum of the patient using the navigated inserter construct; setting a trajectory of the navigated inserter construct based on the navigation guidance; impacting the navigated inserter construct along the trajectory; and removing the navigated inserter construct from the acetabular shell.

In certain embodiments, assembling the navigated inserter construct comprises affixing a tracking array to an inserter, wherein the tracking array is adapted for tracking a pose of the navigated inserter construct by the camera tracking system. In further embodiments, assembling the navigated inserter construct further comprises attaching the acetabular shell to the navigated inserter construct, wherein the acetabular shell is of a selected size.

In certain embodiments, the trajectory is set based on one or more of: a target height, a target inclination angle, a target version angle, a target depth, or a screw location via rotation of the acetabular shell in relation to bone.

Certain embodiments further comprise placing a liner in the acetabular shell after removing the navigated inserter construct therefrom.

Certain embodiments further comprise, prior to placing the liner, inserting a fixation structure into a fixation aperture in the acetabular shell, thereby augmenting fixation of the acetabular shell in the acetabulum.

Certain embodiments further comprise removing one or both of a trial shell and a liner from the acetabulum prior to the inserting.

Certain embodiments further comprise, prior to inserting the navigated inserter construct into the acetabulum, registering a location of a fixation aperture of the acetabular shell to the computer platform.

Certain embodiments further comprise affixing a patient reference array to the patient, wherein the patient reference array comprises a plurality of reference elements, and is adapted for tracking by the camera tracking system.

In certain embodiments, the displaying further comprises displaying the navigation guidance to the user on one or more of a display screen or an extended reality (XR) headset in communication with the computer platform.

A third aspect of the disclosure provides a method for performing a navigation-assisted total hip arthroplasty (THA) procedure, comprising providing a camera tracking system adapted to track one or more tracking arrays, and a computer platform in communication with the camera tracking system; affixing a first tracking array to a pelvis of a patient, wherein the first tracking array comprises a plurality of reference elements adapted for tracking by the camera tracking system; assembling a navigated reamer construct comprising a reamer, a reamer basket of a selected size, and a second tracking array, wherein the second tracking array comprises a plurality of reference elements adapted for tracking a pose of the navigated reamer construct by the camera tracking system; inserting the reamer basket of the navigated reamer construct into an acetabulum of the patient; displaying navigation guidance to a user for the navigated reamer construct; reaming the acetabulum with the navigated reamer construct, based on the navigation guidance displayed to the user; removing the navigated reamer construct from the acetabulum; assembling a navigated inserter construct comprising an inserter, an acetabular shell coupled thereto, and a third tracking array comprising a plurality of reference elements adapted for tracking a pose of the navigated inserter construct by the camera tracking system; displaying navigation guidance to a user for the navigated inserter construct; inserting the acetabular shell into the acetabulum of the patient using the navigated inserter construct; setting a trajectory of the navigated inserter construct based on the navigation guidance; impacting the navigated inserter construct along the trajectory; and removing the navigated inserter construct from the acetabular shell.

Certain embodiments further comprise inputting data indicating the selected size of the reamer basket into the computer platform by using a touch screen or an extended reality (XR) interaction with an XR headset that is in communication with the computer platform, wherein the displaying further comprises displaying the navigation guidance to the user on one or more of a display screen or the XR headset.

These and other aspects, advantages and salient features of the disclosure will become apparent from the following detailed description, which, when taken in conjunction with the annexed drawings, describe embodiments of the disclosure.

### BRIEF DESCRIPTION OF THE DRAWINGS

Aspects of the present disclosure are illustrated by way of example and are not limited by the accompanying drawings.

FIG. 1 is an overhead view of a surgical system arranged during a surgical procedure in a surgical room which includes a camera tracking system for computer assisted navigation during surgery, and a surgical robot for robotic assistance according to some embodiments of the disclosure.

FIG. 2 further illustrates the camera tracking system and the surgical robot of FIGS. 1-2, configured according to some embodiments of the disclosure.

FIG. 3 illustrates a block diagram of a surgical system that includes an extended reality headset, a computer platform, imaging devices, and a surgical robot, which are configured to operate according to some embodiments of the disclosure.

FIG. 4 illustrates a flowchart of a workflow during an intra-operative portion of a total hip arthroplasty (THA) surgery, in accordance with some embodiments of the disclosure.

FIG. 5 illustrates a flowchart of a patient preparation process before registration, in accordance with some embodiments of the disclosure.

FIG. 6 illustrates a radiographic inclination angle measured in the coronal plane of the patient, in accordance with some embodiments of the disclosure.

FIG. 7 illustrates a radiographic version angle measured relative to the coronal plane of the patient, in accordance with some embodiments of the disclosure.

FIG. 8 illustrates different views of landmarks and axes for registration of a functional pelvic plane (FPP) and an anterior pelvic plane (APP) of a patient, in accordance with some embodiments of the disclosure.

FIG. 9 provides a perspective view of a reference array attached to rigid anatomy of a patient, in accordance with some embodiments of the disclosure.

FIG. 10 provides a perspective view of an assembled navigated reamer construct, in accordance with some embodiments of the disclosure.

FIG. 11 provides a perspective view of an assembled navigated reamer construct being inserted into the acetabulum, in accordance with some embodiments of the disclosure.

FIG. 12 provides a flow chart illustrating a workflow for preparation of the acetabulum by reaming with navigation guidance, in accordance with some embodiments of the disclosure.

FIG. 13 provides a perspective view of an assembled navigated inserter construct with an acetabular shell coupled thereto, in accordance with some embodiments of the disclosure.

FIG. 14 provides a perspective view of an assembled navigated inserter construct being inserted into an acetabulum to fixate the shell, in accordance with some embodiments of the disclosure.

FIG. 15 provides a flow chart illustrating a workflow for acetabular shell placement with navigation guidance, in accordance with some embodiments of the disclosure.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION OF THE DISCLOSURE

Surgery systems including computer-assisted navigation capabilities are useful in arthroplasty procedures to provide surgeons with computerized visualizations of how a surgical instrument or other device that is posed relative to a patient correlates to a pose relative to medical images of the patient's anatomy, and how those poses correlate to a pre-operative surgical plan. Camera tracking systems for computer assisted surgery navigation typically use a set of tracking cameras to track a pose of one or more reference elements on the surgical instrument, which may be coupled to a surgical robot or may be operated freehand, i.e., manually, by a surgeon. In either case, during surgery, the surgical instrument may be positioned by the surgeon relative to a patient reference element (or “dynamic reference base” (DRB)) rigidly affixed to the patient. A computer model of a real instrument is associated with a reference element, so that the computer model can be overlaid on registered images of patient's anatomy. The camera tracking system uses the relative poses of the reference elements to determine how the real instrument is posed relative to the patient and to determine how the computer model of the real instrument is to be correspondingly posed as overlaid on the medical images. In this manner, the surgeon can use real-time visual feedback of the relative poses to navigate the surgical instrument during a surgical procedure on the patient. In the context of a total hip arthroplasty (THA) procedure, the surgeon can use real-time visual feedback of the relative poses of a reamer to prepare the acetabulum, and an inserter instrument to insert the shell into the prepared acetabulum.

The present application is related to (1) patent application Ser. No. 15/180,126, filed Jun. 13, 2016 (U.S. Pat. No. 10,842,453), (2) patent application Ser. No. 15/157,444, filed May 18, 2016 (U.S. Pub. No. 2016/0256225), (3) patent application Ser. No. 18/743,685, filed Jun. 14, 2024 (Docket ROBOT.143.0005), (4) patent application Ser. No. 18/743,388, filed Jun. 14, 2024 (Docket ROBOT.143.0002), (5) patent application Ser. No. 18/743,647, filed Jun. 14, 2024 (ROBOT.143.0004), (6) patent application Ser. No. 18/743,615, filed Jun. 14, 2024 (Docket ROBOT.143.0003), (7) patent application Ser. No. 18/770,993, filed Jun. 14, 2024 (Docket ROBOT.146.0002), (8) patent application Ser. No. 18/770,993 (ROBOT.146.0002), and (9) patent application Ser. No. 18/801,924 (Docket ROBOT.148.0002), each of which is incorporated herein by reference.

As noted above, in certain embodiments, the computer navigation-assisted surgical system may be used in connection with a navigated surgical instrument adapted for freehand navigation by the surgeon (or “user”). In other embodiments, the computer navigation-assisted surgical system may include a robotic system (or, “robot” or “surgical robot”) having a serial arm on which an end effector is mounted. The user may hold the end effector or any instruments coupled thereto, to perform surgical operations while watching in real time on a navigation system (e.g., on stand-alone display(s) or an Augmented Reality (AR) headset), and to receive various types of relevant feedback and information associated with a defined plan for and/or progress of the surgical procedure.

Various workflows can be available for use with the system. Such workflows may incorporate preoperative scans or images of the patient (e.g., x-ray or Computerized Tomography (CT)). On the other hand, other workflows may be imageless, and may not require any pre-operative images. Some workflows may incorporate acquisition of intra-operative information about the patient anatomy. In one example, the surgeon may measure key parameters of the bone using a camera tracking system and an appropriate tracked instrument to capture points on patient anatomy. Later, this information, and other intra-operatively-acquired information may be used to plan the implant position and orientation with respect to patient anatomy, and to navigate the surgical instruments, either freehand or via the surgical robot during the surgical procedure.

In some workflows, the surgeon may rigidly attach a reference element to one or more bones, where the reference element includes fiducials adapted for detection by tracking cameras for computer assisted navigation. The reference element(s) allow tracking of bone position by the navigation system. The reference element(s) can be positioned on the bone and oriented such that they are detectable, e.g., can be seen by the tracking cameras of the navigation system. Once positioned, the reference elements are attached with fixation structures (e.g., screw pins, “crocodile” jaws) on the bone (e.g., pelvis or femur). The reference elements'respective positions and orientations stay rigidly fixed with respect to the bone throughout the procedure.

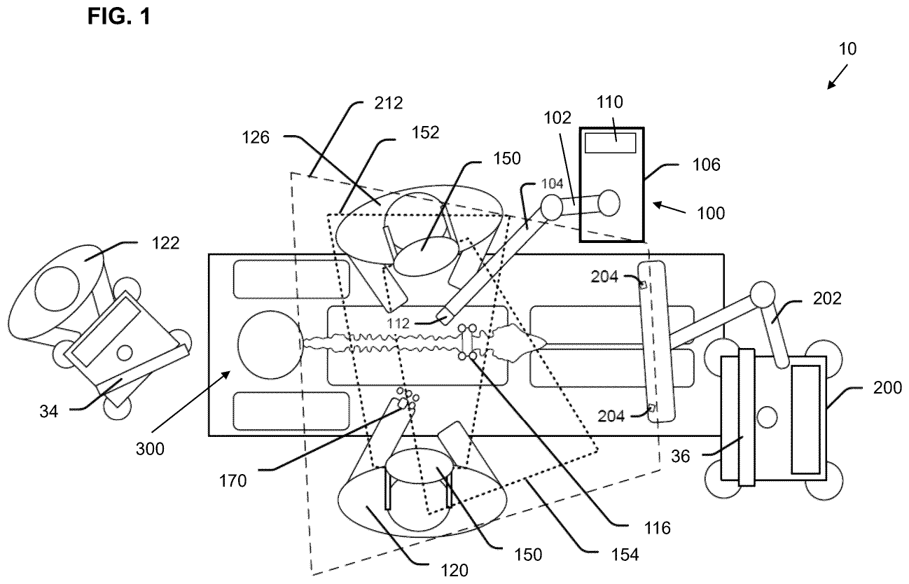

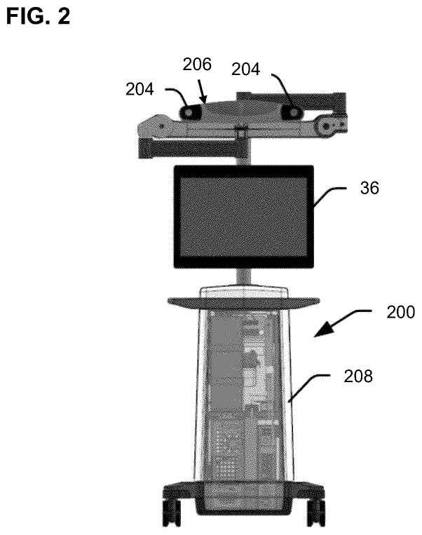

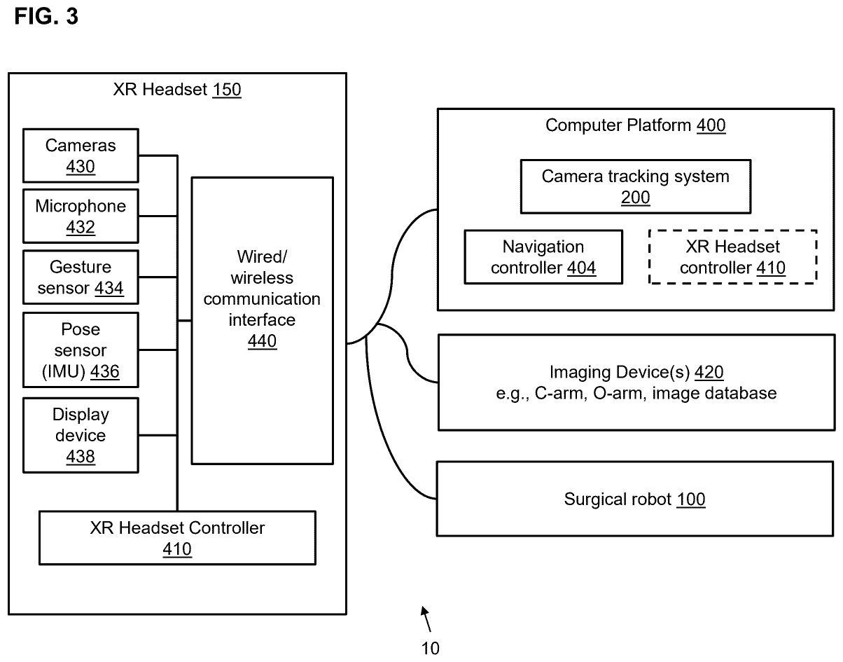

Turning to the figures, FIG. 1 is an overhead view of a computer navigation-assisted surgical system 10 arranged during a surgical procedure in a surgical or operating room. The system 10 includes a camera tracking system 200 for computer assisted navigation during surgery and may further include a surgical robot 100 for robotic assistance according to certain embodiments. FIG. 2 further illustrates the camera tracking system 200 configured according to some embodiments. FIG. 3 illustrates a block diagram of a surgical system 10 that includes an extended reality (XR) headset 150, a computer platform 400, imaging devices 420, and the surgical robot 100 which are configured to operate according to some embodiments. In certain embodiments, the computer platform 400 including the camera tracking system 200 may be used in combination with the XR headset 150 and/or the imaging device(s) 420 to assist the surgeon in performing freehand procedures, i.e., use of the surgical robot 100 may be omitted.

The camera tracking system 200 (FIGS. 1-3) in some cases includes an intraoperative imaging system, that can include distinct imaging modalities. These imaging modalities may include one or more of fluoroscopy, 2D Radiography, and Cone-beam computed tomography (CBCT). Fluoroscopy is a medical imaging technique that shows a continuous X-ray image on a monitor, much like an X-ray movie. 2D Radiography is an imaging technique that uses X-rays to view the internal structure of a non-uniformly composed and opaque object such as the human body. CBCT (or, cone beam 3D imaging or C-arm CT), is a medical imaging technique consisting of X-ray computed tomography where the X-rays are divergent, forming a cone. The camera tracking system 200 is capable of: (1) capturing three-dimensional (3D) images (e.g., CT, CBCT, MCT, PET, Angiogram, MRI, ultrasound, etc.), (2) capturing two-dimensional (2D) images (e.g., fluoroscopy, digital radiography, ultrasound, etc.), and (3) containing an integrated or detachable navigation array having tracking markers (e.g., NIR retroreflective, NIR LED, visible, etc.), which is calibrated to the image space of the 2D and 3D images.

The system 10 is capable of: (1) using registered 2D and/or 3D images for surgical planning, navigation, and guidance in a variety of workflows (e.g., intraoperative 3D, intraoperative 2D, preoperative 3D to 2D, and intraoperative 3D to 2D, etc.); and (2) containing a camera tracking system 200 capable of tracking markers (e.g., NIR retroreflective, NIR LED, visible, etc.). In some cases, as noted herein, a dynamic reference base (DRB) (or patient reference array) 116 is (1) capable of rigidly attaching to the patient anatomy, and (2) contains an array of tracking markers (e.g., NIR retroreflective, NIR LED, visible, etc.).

The XR headsets 150 may be configured to augment a real-world scene with computer-generated XR images while worn by personnel in the operating room. The XR headsets 150 may be configured to provide an augmented reality (AR) viewing environment by displaying the computer-generated XR images on a transparent display screen that allows light from the real-world scene to pass therethrough for combined viewing by the user. Alternatively, the XR headsets 150 may be configured to provide a virtual reality (VR) viewing environment by preventing or substantially preventing light from the real-world scene from being directly viewed by the user while the user is viewing the computer-generated AR images on a display screen. The XR headsets 150 can be configured to provide both or either of AR and VR viewing environments. Thus, the term XR headset encompasses both or either of an AR headset or a VR headset.

With continuing reference to FIGS. 1-3, the surgical robot 100 may include, for example, one or more robot arms 102, 104, a display 110, an end effector 112, for example, including a guide tube and an end effector reference element which can include one or more tracking fiducials.

A patient reference element (or dynamic reference base (DRB)) 116 (shown in FIG. 1) may include a plurality of reference elements, e.g., tracking fiducials, and is secured directly to the patient 300. For example, a navigated pelvis DRB marker array may be placed intra-incision or extra-incision with the help of cortical pins drilled into the pelvic bone. In some embodiments, the DRB is oriented to be visible by the tracking camera(s) 204 (e.g., a stereoscopic tracking camera) installed on the camera tracking system 200 and/or the XR headset 150. Additionally, a reference element 170 may be attached to or formed on an instrument, surgical tool, surgical implant device, etc., which may also be detectable by the tracking camera(s) 204 of camera tracking system 200 and/or the XR headset 150.

As noted, the camera tracking system 200 includes tracking cameras 204, e.g., two tracking cameras 204 as shown in FIGS. 1-2, which may be spaced apart to provide stereo cameras configured with partially overlapping fields-of-view (FOV). The camera tracking system 200 can have any suitable configuration of arm(s) 202 to move, orient, and support the tracking cameras 204 in a desired location, and may contain at least one processor operable to track the location of an individual fiducial and pose of an array of fiducials of a reference element.

As used herein, the term “pose” refers to the location (e.g., along three orthogonal axes, e.g., the x-, y-, and z-axes) and/or the rotation angle (e.g., about the three orthogonal axes) of fiducials (e.g., of a DRB) relative to another fiducial (e.g., surveillance fiducial) and/or to a defined coordinate system (e.g., camera coordinate system, navigation coordinate system, etc.). A pose may therefore be defined based on only the multidimensional location of the fiducials relative to another fiducial and/or relative to the defined coordinate system, based on only the multidimensional rotational angles of the fiducials relative to the other fiducial and/or to the defined coordinate system, or based on a combination of the multidimensional location and the multidimensional rotational angles. The term “pose” therefore is used to refer to location, rotational angle, or combination thereof of, e.g., an instrument reference element 170 (e.g., FIGS. 1, 10, and 11), a patient reference element 116 (e.g., FIGS. 1, 9, 11, and 14), or the like.

The tracking cameras 204 may include, e.g., infrared cameras (e.g., bifocal or stereophotogrammetric cameras) operable to identify, for example, active and passive tracking fiducials for single fiducials (e.g., a surveillance fiducial) and reference elements which can be formed on or attached to the patient 300 (e.g., patient reference element or DRB 116), XR headset(s) 150 worn by a surgeon 120 and/or a surgical assistant 126, instruments such as a driver or an inserter (e.g., reference element 170), etc. in a given measurement volume of a camera coordinate system while viewable from the perspective of the tracking cameras 204. The tracking cameras 204 may scan the given measurement volume and detect light that is emitted or reflected from the fiducials in order to identify and determine locations of individual fiducials and poses of the reference elements in three-dimensions. For example, active reference elements may include infrared-emitting fiducials that are activated by an electrical signal (e.g., infrared light emitting diodes (LEDs)), and passive reference elements may include retro-reflective fiducials that reflect infrared light (e.g., they reflect incoming IR radiation into the direction of the incoming light), for example, emitted by illuminators on the tracking cameras 204 or other suitable device.

The XR headsets 150 may each include tracking cameras (e.g., spaced apart stereo cameras) that can track the location of a surveillance fiducial and poses of reference elements within the XR camera headset fields of view (FOVs) 152 and 154, respectively. Accordingly, as illustrated in FIG. 1, the location of the surveillance fiducial and the poses of reference elements on various objects such as, e.g., instrument reference element 170 and patient reference element 116, can be tracked while in the FOVs 152 and 154 of the XR headsets 150 and/or a FOV 212 of the tracking cameras 204.

FIG. 1 illustrates a potential configuration for the placement of the camera tracking system 200 and the surgical robot 100 in an operating room environment. In certain embodiments, computer-assisted navigated robotic surgery can be provided by the surgical robot 100; and the camera tracking system 200 may control the XR headsets 150 and/or other displays 34, 36, and 110 to display surgical procedure navigation information. In other embodiments, computer-assisted navigated freehand surgery can be performed by the surgeon with the aid of the camera tracking system 200, which may control the XR headsets 150 and/or other displays 34, 36 displaying surgical procedure navigation information.

The camera tracking system 200 may operate using tracking information and other information provided by multiple XR headsets 150 such as inertial tracking information and optical tracking information (frames of tracking data). The XR headsets 150 operate to display visual information and may play-out audio information to the wearer. This information can be from local sources (e.g., the surgical robot 100 if present), imaging devices 420 (FIG. 3), remote sources (e.g., patient medical image database), and/or other electronic equipment. The camera tracking system 200 may track fiducials in 6 degrees-of-freedom (6 DOF) relative to three axes of a 3D coordinate system and rotational angles about each axis. The XR headsets 150 may also operate to track hand poses and gestures to enable gesture-based interactions with “virtual” buttons and interfaces displayed through the XR headsets 150, and can also interpret hand or finger pointing or gesturing as various defined commands. Additionally, the XR headsets 150 may have a 1-10× magnification digital color camera sensor called a digital loupe. In some embodiments, one or more of the XR headsets 150 are minimalistic XR headsets that display local or remote information but include fewer sensors and are therefore more lightweight.

An “outside-in” machine vision navigation bar 206 supports the tracking cameras 204 and may include a color camera. The machine vision navigation bar 206 generally has a more stable view of the environment because it does not move as often or as quickly as the XR headsets 150 while positioned on wearers'heads. The patient reference element (or, DRB) 116 is generally rigidly attached to the patient 300 with stable pitch and roll relative to gravity. This local rigid patient reference 116 can serve as a common reference for reference frames relative to other tracked elements, such as a reference element on the end effector 112 of the robot 100 (when present), instrument reference element 170, and reference elements on the XR headsets 150.

In some example embodiments, the XR headset(s) 150 can be controlled to dynamically display an updated graphical indication of the pose of a surgical instrument so that the user, e.g. surgeon 120, can be aware of the pose of the surgical instrument at all times during the procedure.

In some embodiments, at the end of the end effector 112 of the robot 100, instruments are connected to perform operations such as resection, reaming, broaching, and implant placement and adjustment. In other embodiments, these instruments are operated in a freehand manner by the surgeon to perform the same operations without the aid of the surgical robot.

In certain embodiments, the surgical robot 100 may be positioned near or next to patient 300 as shown in FIG. 1. The robot 100 can be positioned at any suitable location near the patient 300 depending on the area of the patient 300 undergoing the surgical procedure. The camera tracking system 200 may be separate from the robot system 100 and positioned at the foot of patient 300. This location allows the tracking camera 200 to have a direct visual line of sight to the surgical area, e.g., the hip area. In the configuration shown in FIG. 1, the surgeon 120 may be positioned alongside the patient 300 such that the surgeon 120 and any instruments being utilized or manipulated by the surgeon 120 are visible in the FOV 212 of the cameras 204, and the display 36 is visible to the surgeon 120. A surgical assistant 126 may be positioned across from the surgeon 120. If desired, the locations of the surgeon 120 and the assistant 126 may be reversed. An anesthesiologist 122, nurse, or scrub tech can operate equipment which may be connected to display information from the camera tracking system 200 on a display 34 (FIG. 1).

Fiducials of reference elements can be formed on or connected to robot arms 102 and/or 104, the end effector 112, and/or a surgical instrument (e.g., instrument reference element 170) to enable tracking of poses in a defined coordinate system, e.g., such as in six degrees of freedom (DOF) along three orthogonal axes and rotation about the axes. The reference elements 116, 170 enable each of the marked objects (e.g., the patient 300, and the surgical instruments, respectively) to be tracked by the tracking camera 200, and the tracked poses can be used to provide navigated guidance during a surgical procedure, including a manually performed surgical procedure, and/or to control movement of the surgical robot 100 for guiding the end effector 112 and/or an instrument manipulated by the end effector 112.

Referring to FIG. 1 the surgical robot 100 may include a display 110, upper arm 102, lower arm 104, and end effector 112. Cabinet 106 may house electrical components of surgical robot 100 including, but not limited to, a battery, a power distribution module, a platform interface board module, and a computer. As shown in FIGS. 1-2, the camera tracking system 200 may include a display 36, tracking cameras 204, arm(s) 202, a computer housed in cabinet 208, and other components.

In computer assisted navigated surgeries, perpendicular 2D scan slices, such as axial, sagittal, and/or coronal views of patient anatomical structure are displayed to enable user visualization of the patient's anatomy alongside the relative poses of surgical instruments. An XR headset 150 or other display such as, e.g., display 36 can be controlled to display one or more 2D scan slices of patient anatomy along with a 3D graphical model of anatomy. The 3D graphical model may be generated from a 3D scan of the patient, e.g., by a CT scan device, and/or may be generated based on a baseline model of anatomy which isn't necessarily formed from a scan of the patient.

### Example Surgical System

FIG. 3 illustrates a block diagram of a surgical system 10 that includes a computer platform 400 including, inter alia, the camera tracking system 200, imaging device(s) 420, and XR headset(s) 150, and an optional surgical robot 100, which are configured to operate as described herein, according to some embodiments.

The imaging device(s) 420 may include a C-arm imaging device, an O-arm imaging device, other imaging device, and/or a patient image database of 2D and/or 3D images. The XR headset 150 provides a human interface for performing navigated surgical procedures. The XR headset 150 can be configured to provide functionalities, e.g., via the computer platform 400, that include without limitation any one or more of: identification of hand gesture-based commands, and display of XR graphical objects on a display device 438 of the XR headset 150 and/or another display device. The display device 438 may include a video projector, flat panel display, etc. The user may view the XR graphical objects as an overlay anchored to particular real-world objects viewed through a see-through display screen. The XR headset 150 may additionally or alternatively be configured to display on the display device 438 video streams from cameras mounted to one or more XR headsets 150 and other cameras.

Electrical components of the XR headset 150 can include a plurality of cameras 430, a microphone 432, a gesture sensor 434, a pose sensor (e.g., inertial measurement unit (IMU)) 436, the display device 438, and a wireless/wired communication interface 440. The cameras 430 of the XR headset 150 may be visible light capturing cameras, near infrared capturing cameras, or a combination of both.

The cameras 430 may be configured to operate as the gesture sensor 434 by tracking for identification user hand gestures performed within the field-of-view (e.g., FOV 152 or FOV 154, shown in FIG. 1) of the camera(s) 430. Alternatively, the gesture sensor 434 may be a proximity sensor and/or a touch sensor that senses hand gestures performed proximately to the gesture sensor 434 and/or senses physical contact, e.g., tapping on the sensor 434 or its enclosure. The pose sensor 436, e.g., IMU, may include a multi-axis accelerometer, a tilt sensor, and/or another sensor that can sense rotation and/or acceleration of the XR headset 150 along one or more defined coordinate axes. Some or all of these electrical components may be contained in a head-worn component enclosure or may be contained in another enclosure configured to be worn elsewhere, such as on the hip or shoulder.

As explained above, the surgical system 10 includes the camera tracking system 200 which may be connected to a computer platform 400 for operational processing and which may provide other operational functionality including a navigation controller 404 and/or an XR headset controller 410. The surgical system 10 may further optionally include the surgical robot 100 in certain embodiments. The navigation controller 404 can be configured to provide visual navigation guidance to a user, e.g., a surgeon, for moving and positioning a surgical instrument relative to patient anatomical structure based on a surgical plan, e.g., from a surgical planning function, defining where a surgical procedure is to be performed using the surgical instrument on the anatomical structure and based on a pose of the anatomical structure determined by the camera tracking system 200. The navigation controller 404 may be further configured to generate navigation information based on a target pose for a surgical tool, a pose of the anatomical structure, and a pose of the surgical tool and/or an end effector 112 of the surgical robot 100. The navigation information may be displayed through the display device 438 of the XR headset 150 and/or another display device to indicate where the surgical instrument, e.g., a reamer or inserter, and/or the end effector 112 of the surgical robot 100 if applicable, should be moved to perform a surgical procedure according to a defined surgical plan.

The electrical components of the XR headset 150 can be operatively connected to the electrical components of the computer platform 400 through the wired/wireless interface 440. The electrical components of the XR headset 150 may be operatively connected, e.g., through the computer platform 400 or directly connected, to various imaging devices 420, e.g., the C-arm imaging device, the O-arm imaging device, other imaging device(s), the patient image database, and/or to other medical equipment through the wired/wireless interface 440.

The surgical system 10 may include a XR headset controller 410 that at least partially resides in the XR headset 150, the computer platform 400, and/or another system component connected via wired cables and/or wireless communication links. Various functionality may be provided by software executed by the XR headset controller 410. The XR headset controller 410 is configured to receive information from the camera tracking system 200 and the navigation controller 404, and to generate an XR image based on the information for display on the display device 438.

The XR headset controller 410 can be configured to operationally process frames of tracking data from the cameras 430 (tracking cameras), signals from the microphone 432, and/or information from the pose sensor 436 and the gesture sensor 434, to generate information for display as XR images on the display device 438 and/or for display on other display devices for user viewing. Thus, the XR headset controller 410 as illustrated as a circuit block within the XR headset 150 is to be understood as being operationally connected to other illustrated components of the XR headset 150 but not necessarily residing within a common housing or being otherwise transportable by the user. For example, the XR headset controller 410 may additionally or alternatively reside within the computer platform 400 which, in turn, may reside within the cabinet 330 of the camera tracking system 200, the cabinet 106 of the surgical robot 100, etc.

### Exemplary Patient Registration Workflows

Various workflows are provided to register the patient in the tracking space of the navigation system described herein above. Patient registration can include matching the patient anatomy with a numeric representation of the corresponding bone, such as a three-dimensional (3D) model of the bone. The bone representation may be constructed from, e.g., a set of CT images (CT workflow), a set of fluoroscopy images, or based on a generic bone model (imageless workflow). In some embodiments of the present disclosure, the system 10, e.g., computer platform 400, may perform one of a number of available workflows to register a patient to the surgical system 10 prior to surgery. The workflows may further include isolating a target area for the surgical procedure from non-target surgical areas.

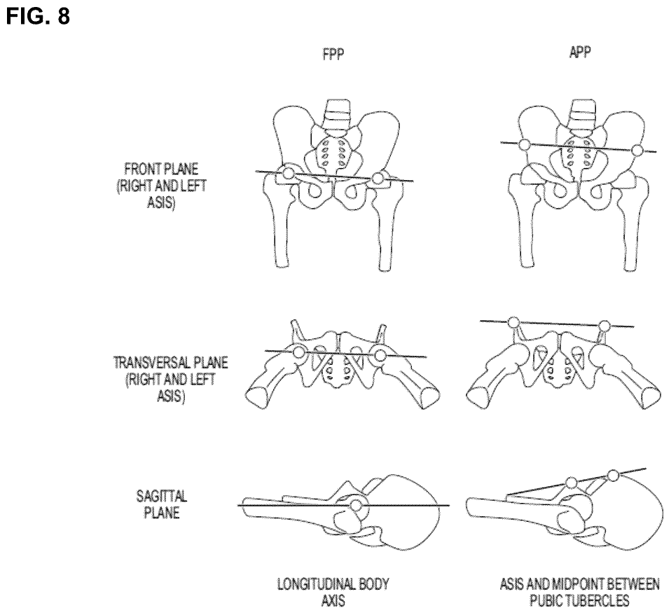

In one embodiment, the workflow may be an imageless workflow in which no pre-operative images are used. Instead, information about the patient anatomy in the operating room (OR) can be obtained by the surgeon measuring key parameters of the patient's bone using the system as described herein. For example, the computer platform 400 of the system 10 operates to identify the locations of landmarks (e.g., points, axes, and/or surfaces) on the bone and register the locations either concurrently with the identification or thereafter. The locations can be used to define reference plane(s) (e.g., anterior pelvic plane (APP) and/or functional pelvic plane (FPP)) (FIG. 8) which, in turn, are used to plan placements of implants and to navigate surgical instruments, either freehand or with the aid of robot 100, during THA surgical procedures.

In some embodiments, the imageless workflow may be used in the initial patient assessment. For example, the surgeon may assess the patient's mobility and health status with assistance from sensors (e.g., sensors made by Globus Medical which are attached to the leg), physical exercises, and/or clinical surveys to determine if THA is recommended. Gathered data may then be stored and processed by the system before being analyzed by the surgeon to facilitate a final decision. Subsequently, the data may be reused by an application (e.g., surgery planning application by Globus Medical) to establish the most appropriate implant surgical plan.

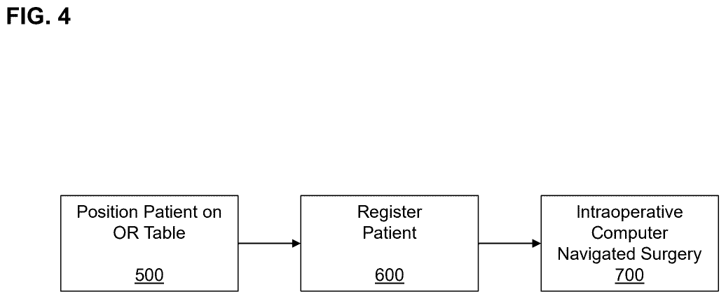

FIG. 4 illustrates a flowchart for a workflow during an intra-operative portion of a THA surgery, in accordance with some embodiments of the present disclosure. In some embodiments, after positioning the patient on the operating room table (process 500), some of the operations discussed above and below may be performed during process 600 to register a patient and before another process 700 for intraoperative computer navigated surgery. In the case of a hip, a pelvis or acetabulum of the patient is registered in the tracking coordinate system of the camera tracking system 200. In one embodiment, the registration is done in an imageless modality without the use of any medical images such as X-rays or CT images from an imaging device. As noted herein, in other embodiments, registration is performed using one or more pre-operative X-ray images and/or CT images.

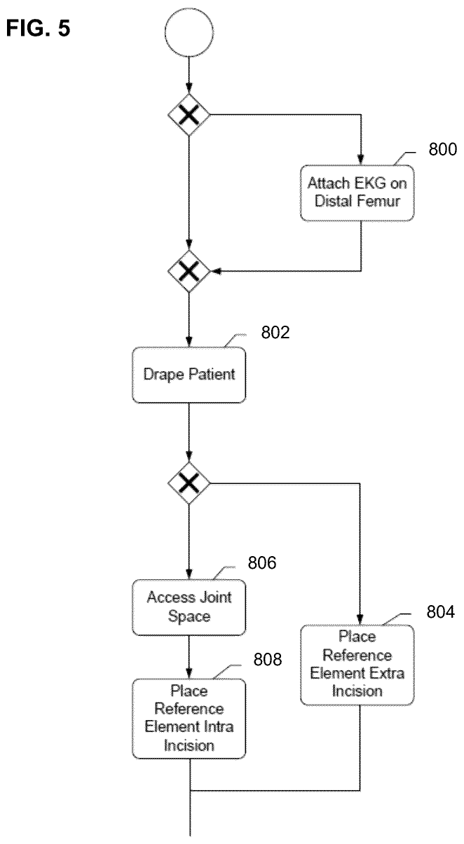

FIG. 5 illustrates a flowchart of a patient preparation process before registration, in accordance with some embodiments of the present disclosure. The patient preparation process may begin with a patient being positioned in a lateral or supine position on the OR table. The patient's body is prepared for registration. Optionally, in process 800, an EKG/ECG patch electrode is attached on or adjacent a distal end of the patient's femur. The EKG/ECG patch electrode may be placed on the center of the patella or slightly inferior to the center. In some embodiments, the patch location is in line with the anatomic axis of the femur. This patch may be used to acquire the most distal point of the femur under the drape at a later stage. This patch may also be used to track the femur in space (e.g., when the patient's leg is moved during surgery) and may also be used to assist in measuring the patient's leg length. However, in some embodiments, this operation (process 800) is skipped.

In some embodiments, the EKG/ECG patch electrode includes an adhesive patch that is removably attachable to the patient. In some embodiments, the patch may be black or dark to be more visible to the tracking camera. In other embodiments, the patch and patch electrodes are not visible by the tracking camera as they are under a drape. The patch geometry (like a nipple) will help the surgeon to always touch a single point on or adjacent the distal part of the femur (anterior patella region) with a navigated stylus/instrument which is trackable by the tracking camera. This ensures that the surgeon always collects the same point to measure the leg length or medio-lateral offset.

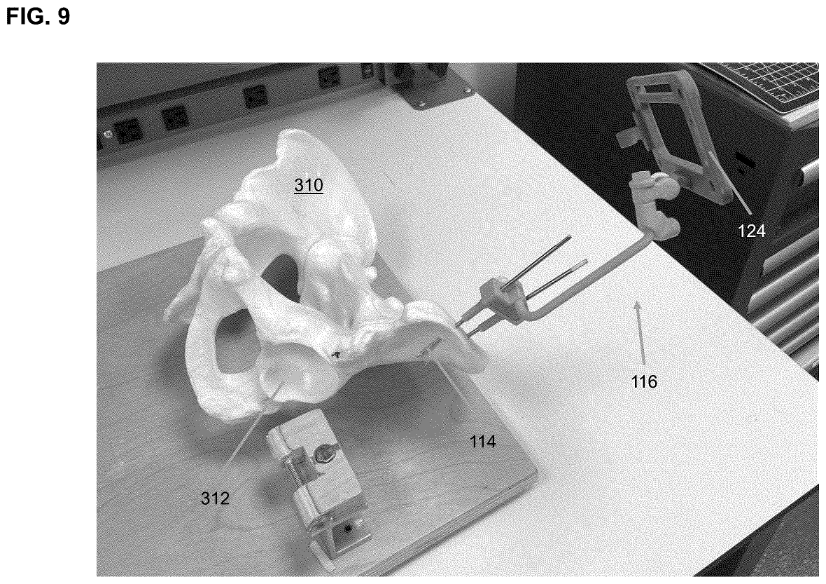

In process 802, the patient body is draped. Then, depending on the surgeon's technique, the navigated pelvis DRB, e.g., DRB 116 (FIG. 9), is placed intra-incision (processes 806-808) or extra-incision (process 804) with the help of cortical pins drilled into the pelvic bone. In some embodiments, the DRB 116 is oriented to be visible to the tracking camera(s), e.g., stereoscopic tracking cameras 204 installed on the camera tracking system 200 (FIG. 1) or the XR headset 150 (FIG. 1). In one embodiment, the operation to place the DRB intra-incision includes using the system 10 to track and navigate access to the joint space (process 806) and placing the reference element intra-incision. In an alternative embodiment, the reference element 116 is placed extra-incision (process 804) and the system does not necessarily need to be used to track and navigate access to the joint space.

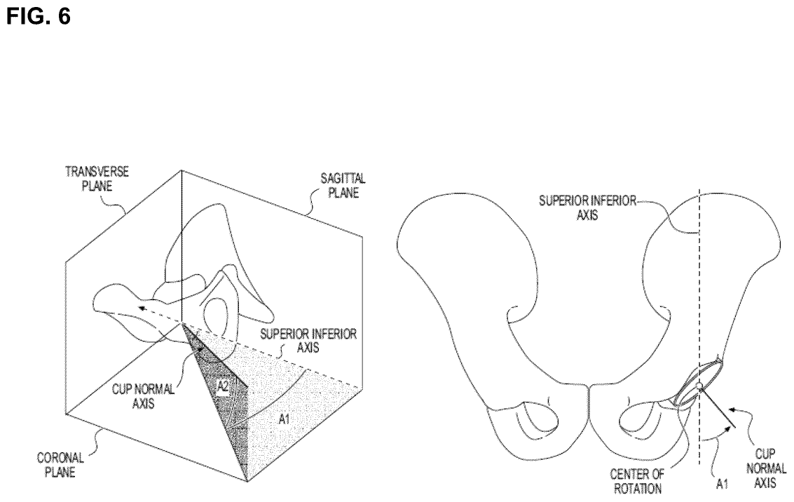

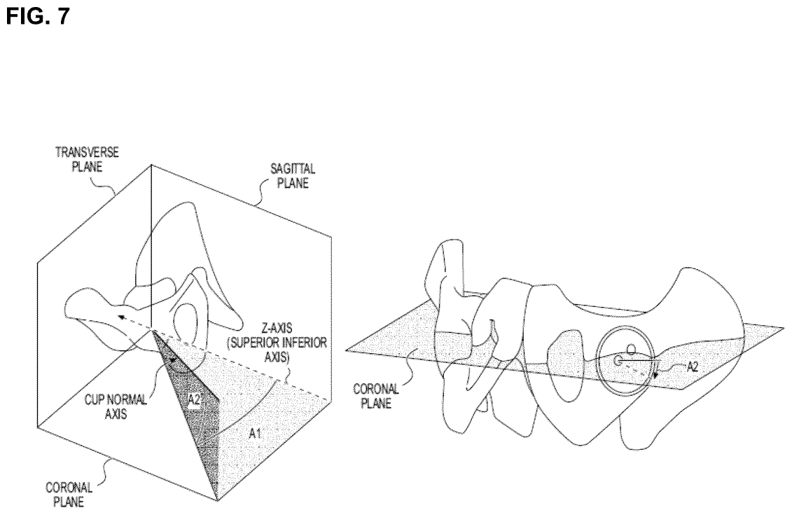

After the reference element, e.g. DRB 116, has been placed intra-incision or extra-incision, data points and axes can be collected on the patient anatomy with the assistance of navigated instruments and using the pelvis DRB coordinate system as a spatial reference. In addition to this, two pelvic reference planes can be established to plan placement of implants by measuring angular deviations such as inclination and version of the acetabular cup implant as shown in FIGS. 6-7.

FIG. 6 illustrates a radiographic inclination angle measured in the coronal plane of the patient, in accordance with some embodiments of the present disclosure. In some embodiments, the surgeon may use a navigated instrument to palpate or paint the surface of the acetabular cavity of the patient to determine a center of rotation of the acetabulum. FIG. 7 illustrates a radiographic version angle measured relative to the coronal plane of the patient, in accordance with some embodiments of the present disclosure. The two pelvic reference planes (or coronal or frontal planes), the anterior pelvic plane (APP) and functional pelvic plane (FPP), are determined or defined using different landmarks and axes as shown on FIG. 8.

During a patient registration procedure, landmarks used to register patient anatomy can be extracted using either single point palpation collection or surface painting (resulting in a point cloud of locations). FIG. 8 illustrates different views of landmarks and axes for registration of the FPP and APP of a patient, in accordance with some embodiments of the present disclosure. The landmarks and axes used to register the APP and FPP planes are described in more detail in U.S. patent application Ser. No. 18/430,077 (Docket No. ROBOT.134.0002), previously incorporated by reference herein.

Further, U.S. patent application Ser. No. 18/430,077 (Docket No. ROBOT.134.0002) discloses processes for registration of a pelvic acetabulum of a patient (including painting the acetabular cavity), in accordance with various embodiments of the present disclosure. For example, to define the APP and FPP origins, the pelvic acetabular center of rotation can be determined after removing the femoral head of the patient from the acetabular cavity. The acetabular cavity may be made accessible by cutting the femoral neck and by removing the femoral head from the acetabular cavity. In some embodiments, a cork screw instrument may be used to remove the femoral head from the acetabular cavity.

The surface of the acetabular cavity can then be painted using a navigated instrument such as, e.g., a stylus. For example, the surgeon may use the navigated instrument (e.g., stylus) to palpate the surface of the acetabular cavity, as the tracking camera measures the position of a ball on the end of the stylus in a continuous way. This process provides a cloud of points for the measured positions (locations). At the same time, the tracking camera may also monitor and track the pose of the patient DRB 116 attached to the pelvis such that the pose of the stylus can be tracked relative to the pose of the patient DRB. Alternatively, the surgeon may subsequently measure a predefined number or percentage of points by palpating them one-by-one. Based on these points and the tracking data of the stylus and patient DRB 116, the center of rotation of the pelvic acetabular cavity is determined. Additionally, based on these points, the surface of the acetabular cavity may be registered in the system and/or a 3D model may be generated or modified based on these points. Next, the acetabular cavity shape can be recreated (e.g., in a 3D model) by the system based on the measured cloud of points and using other algorithms, e.g., for outlier removals and surface fitting.

While certain imageless approaches are described herein and in U.S. patent application Ser. No. 18/430,077 (Docket No. ROBOT.134.0002), previously incorporated by reference herein, other example methods of performing imageless and image-based registration of the pelvis to the tracking coordinate system of the tracking system (e.g. optical coordinate system). These methods may also be used to, e.g., determine a native center of rotation of the acetabulum, derive or define an FPP, and derive or define an APP. Registration may allow a navigation system or robotic system to track any navigated instrument or end effector 112 or any tool attached to the end effector 112 relative to the pelvis as tracked by a patient dynamic reference base 116 attached to the pelvis. Various registration methods described herein can be combined in keeping with various disclosed embodiments.

For example, in one imageless method, an APP is derived by either touching various known points (e.g., left and right anterior superior iliac spine (ASIS) and pubic symphysis) with a navigated instrument, or by a physician lining up a plane or axis defined by the navigated instrument along or parallel to the APP. With the center of rotation and APP determined, the system (either a navigation system or a combined navigation and robot system 100) has sufficient information to register the acetabulum in the coordinate system (e.g., optical coordinate system) of the camera tracking system 200. In both of the above-noted example methods, the tracking system may be constantly monitoring and tracking the pose of the patient DRB 116 attached to the pelvis while also tracking the navigated instrument (e.g., stylus) such that the pose of the instrument can be tracked relative to the pose of the patient DRB, at least for purposes of registering the pelvis relative to the patient DRB 116 in the tracking coordinate system of the camera tracking system 200.

Some exemplary image-based examples include the use of pre-operative CT images, intra-operative fluoroscopy images, and intra-operative point cloud data acquired via a navigated instrument, as described in patent application Ser. No. 18/743,388 (Docket ROBOT.143.0002), and patent application Ser. No. 18/743,615 (ROBOT.143.0003). Other exemplary image-based registration approaches can be performed using intra-operative fluoroscopy images, as described in patent application Ser. No. 18/743,647 (Docket ROBOT.143.0004). In a further exemplary image-based approach to patient registration, intra-operative fluoroscopy images and intra-operative point cloud data acquired using a navigated instrument may be used, as described in patent application Ser. No. 18/743,685 (Docket ROBOT.143.0005).

It is to be understood herein that although the user interfaces and associated operations are described as being performed in a certain sequence, they may be performed in other sequences while still being within disclosed embodiments. Moreover, it is not necessary that all of the user interfaces and/or described operations be performed. Instead, fewer operations may be performed while still being within disclosed embodiments. Further, additional registration approaches can include image-based and imageless workflows. Combinations of these registration approaches are also possible in keeping with the various disclosed embodiments.

### Computer-Navigation Assisted Reaming

According to embodiments of the present disclosure, the surgical system 10, including various features described herein with reference to FIGS. 1-3, including, e.g., camera tracking system 200, may be used perform various workflows for computer-navigation-assisted surgical processes following registration of the patient, in accordance with workflows described herein as well as those known in the art.

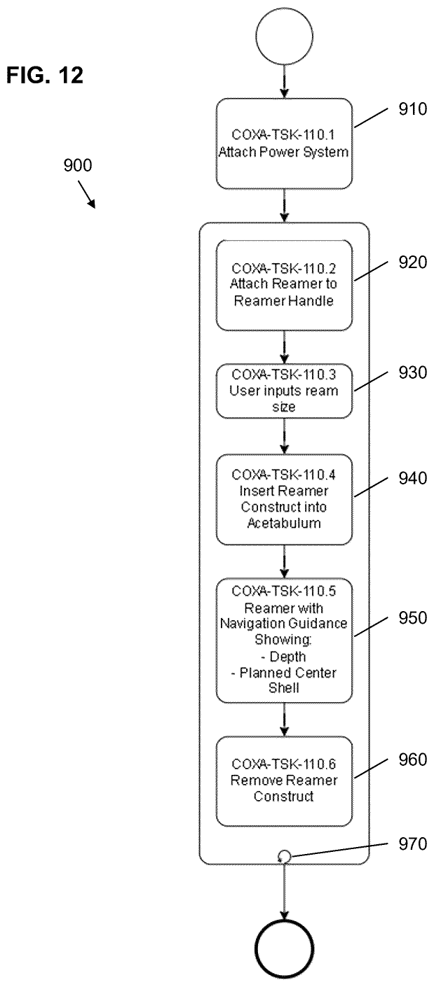

With reference to the depictions of FIGS. 9-11 and the flow diagram of FIG. 12, one such workflow 900 for computer navigation-assisted surgery (FIG. 12) may include methods for preparing one or more bones such as, e.g., the acetabulum, for implant placement during THA surgery.

Preparation of the acetabulum 312 may include reaming the acetabular cavity. One objective of the reaming workflow 900 includes use of the system 10, including camera tracking system 200, to provide navigation guidance to the user, enabling the user (e.g., surgeon 120) to manually guide a navigated reamer construct into the patient's native acetabulum, and expand the diameter thereof to prepare the acetabular cavity for placement of an acetabular implant or “shell.” The system 10, including a computer platform 400, camera tracking system 200, and other components as described herein, is adapted to generate and provide, e.g., display to the user, navigational guidance useful to position a navigated reamer construct and perform reaming according to a defined surgical plan with improved accuracy, as compared to methodologies based on the use of manual guides and/or fluoroscopy to determine appropriate angles and placements of implants during surgery. The surgeon may perform the planned surgical procedure by holding the instrument, e.g., a navigated reamer construct with a reamer basket attached, and watching the procedure processes occur on a display screen such as display 36 and/or via an XR headset 150.

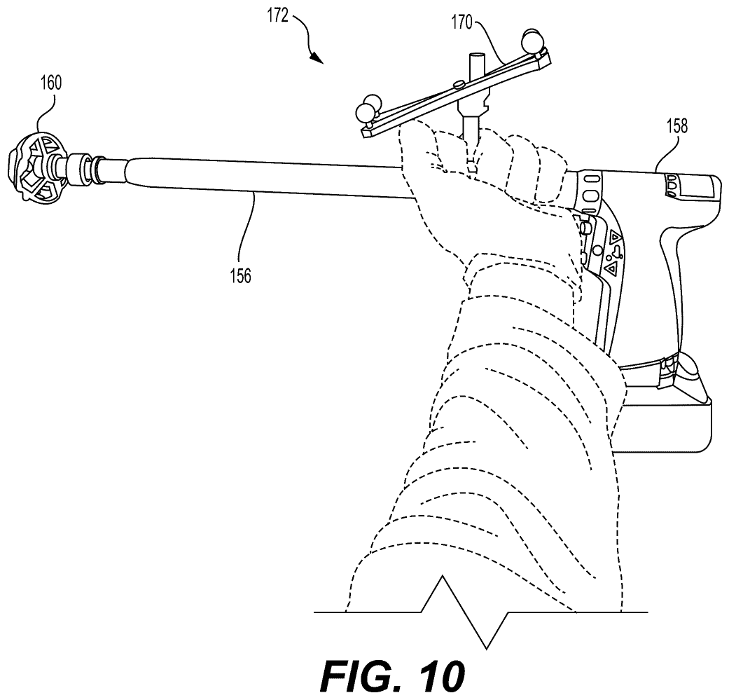

Prior to commencement of workflow 900, or contemporaneously therewith, a instrument tracking array 170 is affixed to a reamer driver 156 (FIG. 10). The tracking array 170 may be adapted to enable tracking of the navigated reamer construct 172 by the camera tracking system 200 as described herein. In particular, the tracking array 170 may include a plurality of reference elements formed thereon or connected thereto, enabling tracking of poses of the navigated reamer construct 172 in a defined coordinate system.

Additionally, a patient reference element, e.g., dynamic reference base (DRB) 116, is affixed to rigid anatomy of the patient, if not already present. The DRB 116 (shown in FIG. 9) may include a tracking array 124 having plurality of tracking fiducials disposed thereon and may be placed intra-incision or extra-incision, and secured directly to the pelvis 310 of the patient via cortical pins 114. The DRB 116 may be oriented and positioned in a pose selected for its visibility by the tracking camera(s) 204 (e.g., a stereoscopic tracking camera) installed on the camera tracking system 200 and/or the XR headset 150 (FIG. 1). In certain embodiments, the DRB 116 may be adjustable, as described in U.S. patent application Ser. No. 18/770,993 (Docket No. ROBOT.146.0002), previously incorporated by reference herein. Regardless of specific embodiment, the presence of DRB 116 enables tracking of poses of the navigated reamer construct 172 relative to the DRB 116 and therefore the patient during the procedure. The tracked poses can then be used by the navigation controller 404 to generate navigated guidance during a surgical procedure for display to the user or surgeon.

Turning to workflow 900, processes 910 through 930 describe further processes in assembling a navigated reamer construct including a reamer basket (or “reamer”). At process 910, the reamer driver 156, previously coupled with a tracking array 170, is operatively coupled with a power system 158, as illustrated in FIG. 10. At process 920, a reamer basket 160 is affixed to a distal end of the reamer driver 156. The reamer basket 160 affixed to the driver 156 may be of a size (e.g., diameter) selected based on, e.g., a defined plan for the surgical procedure, a planned acetabular implant (or “shell”) size, various attributes of patient anatomy, and other factors. Collectively, the reamer driver 156, power supply 158, reamer 160, and reference element 170 may be referred to as a navigated reamer construct 172.

At process 930, the selected size of the reamer basket 160 may be input into a computer-navigated surgery system, e.g., system 10 (FIGS. 1-3). For example, the selected reamer basket size may be input using a touch screen or an extended reality (XR) interaction with an XR headset such as XR headset 150, e.g., a command defined by a particular hand pose or gesture, or an interaction with a virtual button or interface.

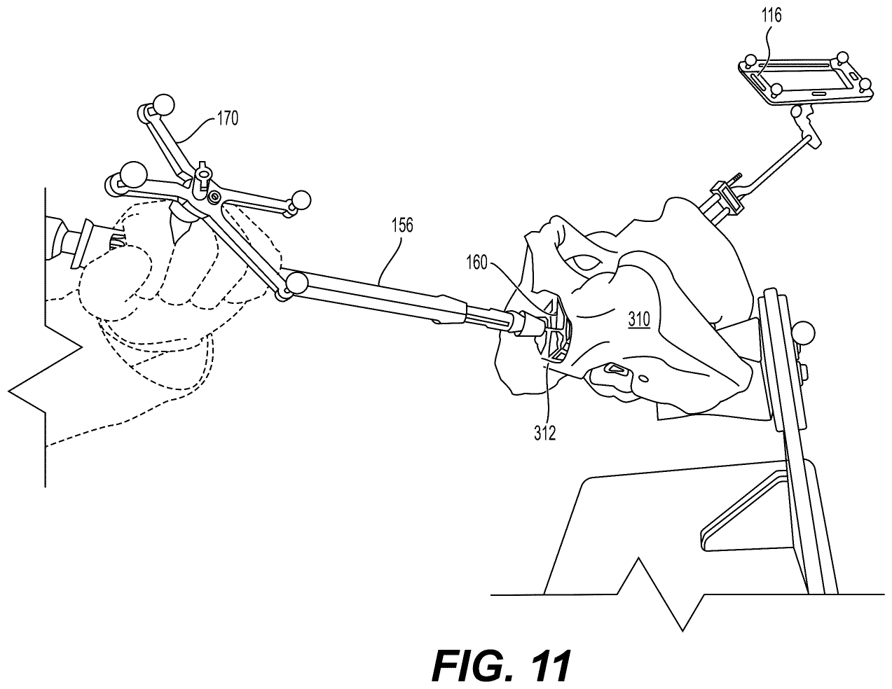

Once assembled, at process 940 the reamer basket 160 of the assembled navigated reamer construct 172 may be inserted into the acetabulum 312 of the patient as shown in FIG. 11. Due to the size constraints of the native acetabulum relative to the reamer basket 160, the center of the reamer basket may in many cases be located outward from the native center of the acetabulum 312 prior to performing reaming.

At process 950, the system 10 may use the input reamer basket size along with additional data relating to, e.g., a pose of the navigated reamer construct 172 relative to the patient (e.g., DRB 116), acquired via the camera tracking system 200, to generate a visual representation of the navigated reamer construct 172 in relation to images of the patient. It is noted that process 950 (or sub-processes thereof) may occur before or concurrently with previously described process such as, e.g., process 940.

The visual representation and other information including navigational guidance for the procedure may be generated by the navigation controller 404 and displayed to a user, for example on a display such as, e.g., display 36 or an XR headset such as, e.g., XR headset 150 (FIG. 1). In various embodiments, the navigation guidance may include, visual navigation guidance for moving and positioning the navigated reamer construct 172 relative to patient anatomy based on one or more of: a pre-defined surgical plan, a pose of the anatomical structure determined by the camera tracking system 200, and a target pose of the navigated reamer construct 172, which may encompass, e.g., a depth and a planned acetabular shell center.

In certain embodiments, the navigational guidance may further include guidance relating to alignment, assisting the user to identify and position the navigated reamer construct 172 along the desired or defined ream trajectory for the eventual reaming. Such alignment guidance may be provided, e.g., when proximity of the reamer basket 160 to the acetabulum 312 is detected by computer platform 400, e.g., navigation controller 404.

As illustrated in FIG. 11, the acetabulum 312 may then be reamed using the navigated reamer construct 172, which may be deployed by the user based on, or in a manner consistent with, the navigational guidance displayed to the user as described herein. For example, when the center of the reamer basket 160 is colocalized with a native center of the acetabulum, and the navigated reamer construct 172 is aligned with a planned ream trajectory, reaming may be performed along the ream trajectory from the native center of the acetabulum to the planned center of the acetabular shell, removing bone tissue that is damaged, e.g., by arthritis or injury. The reamer 160 may further be rotated about a center point thereof during reaming, in order to achieve a smooth, prepared surface within the acetabular cavity, without leaving irregularities, e.g., relatively higher or lower points along the surface. When the center point of the reamer 160 reaches the planned center point of the shell, the computer platform 400 may be operative to provide guidance in the form of an indication to the user to stop the reaming process, so that the center of the reamer 160 does not pass the planned center point of the shell. The indication may be provided visually as described herein, or audibly, e.g., via XR headset 150.

The cooperation of the navigated reamer construct 172 and the system 10, including, e.g., the camera tracking system 200, computer platform 400, and the XR headset 150 (FIG. 3), facilitate the provision to the user of continuous and ongoing feedback regarding the positions and poses of the navigated reamer construct 172 relative to the patient (e.g. DRB 116), and the progress of the reaming workflow in real time or very nearly in real time. This feedback regarding the pose of the navigated reamer construct 172 and the reaming progress allows for improved accuracy in preparing the acetabulum to receive an implant.

In process 960, the navigated reamer construct 172 is removed from the acetabulum 312 after reaming is performed based on the navigational guidance. This may include sub-processes such as, e.g., rotating the navigated reamer construct 172 to a desired alignment to facilitate easy removal from the patient.

In certain embodiments, various processes, e.g., processes 920 through 960, may be performed iteratively in the course of preparing the acetabulum. At the conclusion of performing a first iteration of processes 920 through 960, the acetabulum may be evaluated relative to a preoperative plan or defined plan to assess completeness of reaming. For example, using data acquired by the camera tracking system 200 regarding a pose of the navigated reamer construct 172, dimensions of the reamer basket 160 as previously input, registered images of the patient, and other information, the navigation controller 404 of the computer platform 400 may be configured to evaluate a degree or extent to which reaming according to the defined plan has been executed. In a case in which the evaluation result indicates that the pre-operative plan has been executed, navigation guidance at decision 970 may include a visual or audio indication, e.g. provided to the user via XR headset 150, to conclude the workflow 900. In a case in which the evaluation result indicates that the defined plan has not yet been fully realized, navigation guidance may be provided at decision 970 to perform a second or subsequent iteration of certain processes, e.g., processes 920 through 960. For example, two or more iterations or stages, e.g., two, three, four, or more iterations, may be performed as desired to achieve a satisfactorily prepared acetabular surface for the subsequent performance of workflow 1000 (discussed herein below), or another implant placement workflow as will be understood by those skilled in the art.

In certain cases, a first iteration of processes 920 through 960 may be performed using a first selected size (e.g., diameter) of reamer basket 160. In cases in which decision 970 includes the provision of navigation guidance to perform a subsequent iteration, that subsequent iteration may be performed using the same, first selected size reamer basket 160 or a reamer basket 160 of a second selected size, which may be larger or smaller than the first selected size. In the case that use of a reamer basket 160 of a second selected size is indicated, the reamer basket 160 of the first selected size may be removed from the driver 156 after process 960, and a reamer basket of the second selected size may be affixed to the reamer driver 156 in a second iteration of process 920. Processes 930 through 960 may then be performed as described herein with the reamer basket 160 of the second selected size. Further iterations with further or additional selected sizes of reamer baskets may additionally be performed, which may be based on the navigation guidance provided by the system 10.

In still further embodiments, certain processes of workflow 900, but not all of processes 920 through 960 may be performed iteratively. For example, after a first iteration of processes 920 through 960, completeness of the reaming may be evaluated as described above, resulting in a decision 970 to either conclude the workflow 900 or perform additional processes. In certain embodiments in which the same first selected size of the reamer basket 160 is to be used in a second iteration, the second iteration of process 940 may immediately follow process 960 and decision 970, omitting a second iteration of processes 920 and 930. Any combination of single iteration and multiple iteration phases of reaming may be used as needed to achieve the desired result.

### Computer-Navigation Assisted Insertion

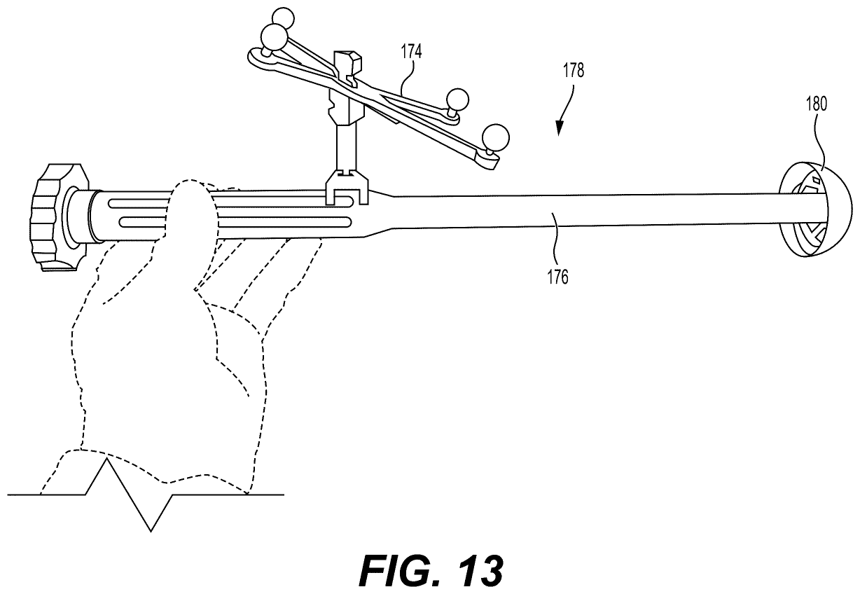

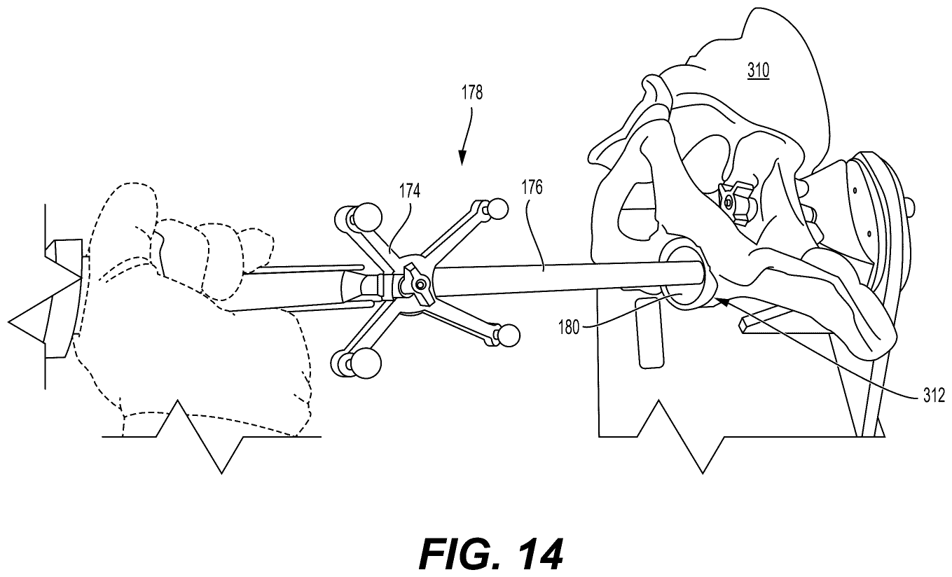

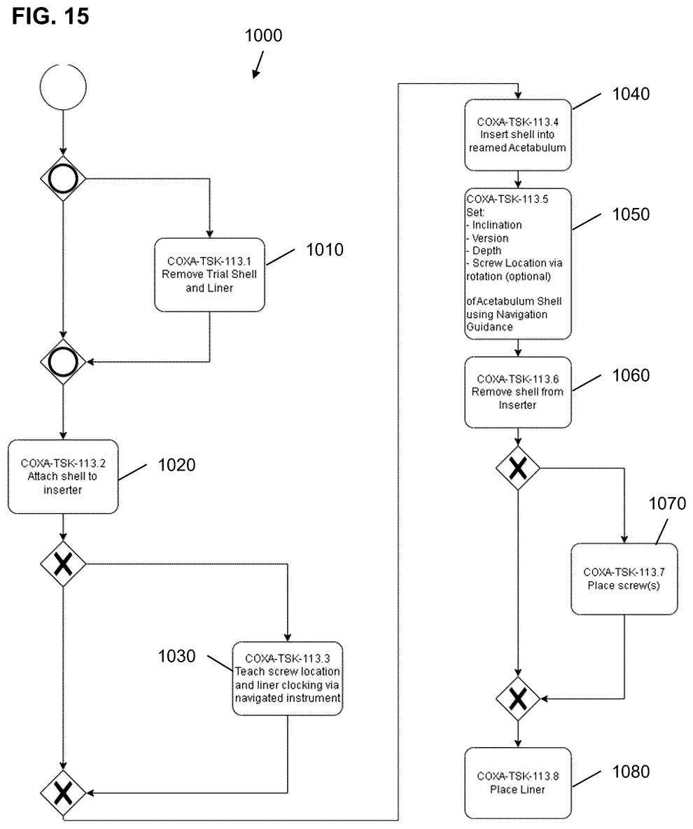

According to further embodiments of the present disclosure, the surgical system 10, including camera tracking system 200 as shown and described herein, may be used to perform further workflows for computer navigation-assisted surgical processes including placement of implants. With reference to the depictions of FIGS. 13-14 and the flow diagram of FIG. 15, one such workflow 1000 (FIG. 15) may include placement of an acetabular shell during THA surgery with the aid of computer-implemented navigation guidance.

FIG. 15 illustrates a workflow 1000 for computer navigation-assisted placement of an acetabular prosthesis (or “shell”). In certain embodiments, workflow 1000 may be performed following workflow 900, in which the acetabulum is prepared for shell placement with the aid of computer-implemented navigational guidance. In such cases, a camera tracking system 200 may already be set up to track all reference arrays used during the placement workflow, and a reference array (e.g. DRB 116) may already be fixedly attached to the pelvis 310 of the patient as shown in FIG. 14. In embodiments in which the acetabulum is not prepared via workflow 900, or is prepared via another workflow not described herein, a patient reference element, e.g., dynamic reference base (DRB) 116, is affixed to rigid anatomy of the patient. The DRB 116 (shown in FIG. 14) may include a plurality of tracking fiducials, or reference elements, and may be placed intra-incision or extra-incision, and secured directly to the pelvis 310 of the patient via cortical pins. The DRB 116 may be oriented and positioned in a pose selected for its visibility by the tracking camera(s) 204 (e.g., a stereoscopic tracking camera) installed on the camera tracking system 200 and/or the XR headset 150 (FIG. 1). The presence of DRB 116 enables tracking of poses of a navigated inserter construct 178 (FIGS. 13-14) relative to the DRB 116 and therefore relative to the patient 310 during the procedure. The tracked poses can then be used by the navigation controller 404 of the computer platform 400 to generate and provide navigated guidance for display to a user during a surgical procedure.

As shown in FIG. 13, assembly of a navigated inserter construct 178 may include affixing a tracking array 174 to the inserter construct 176 in a manner analogous to the affixation of the tracking array 170 to the reamer driver 156, described previously relative to workflow 900. Collectively, the inserter 176 and the tracking array 174 may be referred to herein as a navigated inserter construct 178. The tracking array 174 may be adapted for tracking of a pose of the navigated inserter construct 178 by the camera tracking system 200.

At process 1010 of workflow 1000, the user may, if applicable, remove a trial shell and liner from the patient's acetabulum 312. A trial shell and liner may be present in certain embodiments in which a trialing workflow was performed after the reaming workflow 900 and prior to the implant placement workflow 1000. If no trial shell and liner are present in the acetabulum, or if they have previously been removed, the workflow 1000 may proceed directly to process 1020, in which an acetabular shell 180 of a selected size is coupled to the navigated inserter construct 178. The selected size may be pre-determined by the user as part of a pre-operative plan, and may be selected based on, e.g., fluoroscopy or other imaging.

At an optional process 1030, in certain embodiments, the user may register to the system the location(s) of one or more fixation aperture(s), e.g., screw holes, in the shell 180, as well as liner clocking thereof, using a registration workflow similar to those described herein. For example, in certain embodiments, the fixation aperture and liner clocking features may be taught to the system 10 using a registration workflow in which such landmarks can be extracted using either single point palpation collection or surface painting (resulting in a point cloud of locations) using a navigated instrument, e.g., a navigated stylus. The navigated stylus may include a tracking array that is detectable by the camera tracking system 200, e.g., by cameras 204 (FIG. 2) or cameras 430 of XR headset 150 (FIG. 3), in a manner similar to tracking arrays 170 and 174 of navigated reamer construct 172 and navigated inserter construct 178, respectively. The user may interact with the computer platform 400 by providing inputs via a touch screen or an extended reality (XR) interaction with an XR headset such as XR headset 150 to access the workflow for registering features of the acetabular shell 180. In other embodiments, other registration workflows may be used to similar result. In embodiments in which the locations of the fixation and clocking features are registered to the system 10 in process 1030, the navigation controller 404 of computer platform 400 may be adapted to generate and provide navigational guidance for placement of the fixation structures, e.g., screws later in the workflow 1000, upon placement of the shell (see, e.g., process 1070, discussed below).

The system 10 may use information relating to the pose of the navigated inserter construct 178 relative to the patient (e.g., DRB 116), acquired via the camera tracking system 200, the acquired shell screw location and clocking data if acquired at process 1030, and data (e.g., user-input data) relating to the selected size of the acetabular shell 180, to generate a visual representation of the navigated inserter construct 178 in relation to images of the patient. This visual representation and other information may include navigational guidance for the procedure, which may be displayed to a user, for example on a display such as, e.g., display 36 (FIG. 2) or an XR headset such as, e.g., XR headset 150 (FIG. 3). In various embodiments, the navigation guidance may include, e.g., a target insertion trajectory of the shell 180, which may in turn be based at least in part on a target height, depth, and an orientation, including inclination and version of the shell 180 in relation to the pelvis 310, which may also be displayed to the user.

In various embodiments, the navigation guidance may include, e.g., visual navigation guidance for moving and positioning the navigated inserter construct 178 relative to patient anatomy to realize the target positioning of the shell 180 based on one or more of: a pre-defined surgical plan, a pose of the anatomical structure determined by the camera tracking system 200, and a target pose of the navigated inserter construct 178, and a target position of the shell 180, which may encompass, e.g., an insertion trajectory for the shell 180, and an orientation of the shell 180 in relation to the acetabulum 312.

At process 1040, the navigated inserter construct 178 including the acetabular shell may be inserted into the acetabulum 312. As previously noted, the acetabulum 312 may have been prepared according to workflow 900 described herein, or according to an alternative workflow as will be appreciated by one of skill in the art. In some embodiments, the computer platform 400 may be operative to identify a defined insertion trajectory and display the same, e.g., on a display 36 or on XR headset 150 to guide the surgeon. The user may optionally assess whether any adjustment is needed, e.g., in the planned trajectory or alignment of the inserter, and adjustment may be made before proceeding. This assessment may be aided by visualization and/or navigational guidance generated by the navigation controller 404 of computer platform 400, and which may be displayed, e.g., on a display 36 or on XR headset 150.

At process 1050, the user may set the insertion trajectory of the acetabular shell 180. The trajectory may be set relative to the pelvis 310, e.g., relative to the DRB 116 affixed thereto, and may be set based at least in part on the navigational guidance displayed to the user. The navigational guidance may in turn be based on one or more (e.g., all) of the target or planned inclination angle, version angle, height, and depth of the acetabular shell 180. In embodiments in which the screw location was taught to the system at process 1030, the navigational guidance and positioning of the shell 180 may further include the screw location via rotation.

Upon a determination that the trajectory and alignment of the navigated inserter construct 178, and the planned location of the shell 180 are satisfactory, the user may then impact the shell 180 along the trajectory to fixate the shell 180 in the acetabulum 312, and subsequently confirm that the shell 180 is properly positioned and seated.

At process 1060, the navigated inserter construct 178 is disengaged from the fixated shell 180, and the navigated inserter construct 178 is guided by the user from the joint space. At an optional process 1070, one or more fixation structures, e.g., screws may be placed in the fixation apertures in the shell 180 to augment fixation of the shell 180 in the acetabulum 312. As discussed above, in certain embodiments in which the fixation apertures in the shell 180 were previously registered to the system 10, the computer platform 400 may be operative to provide, e.g., display to the user navigational guidance for placement of the screws in the fixation apertures in the shell 180. In other embodiments, screw placement may be omitted, or performed in another manner, e.g., without navigational guidance.

Following removal of the inserter at process 1060, and also following the placement of screws (if any) at process 1070, a liner may be inserted into the shell at process 1080, for subsequent engagement with a femoral head.

Workflow 1000 for computer navigation-assisted acetabular shell placement provides a number of advantages compared to previous methods. For example, workflow 1000, as assisted by system 10 including computer platform 400, camera tracking system 200, XR headset 150, and the navigated inserter construct 178, provides additional accuracy and ongoing feedback when placing the acetabular shell compared to prior methods. Certain embodiments also provide beneficial confirmation of screw location prior to drilling, when used in combination with a registration workflow as disclosed herein.

### Further Definitions and Embodiments

In the above description of various embodiments of present inventive concepts, it is to be understood that the terminology used herein is for the purpose of describing particular embodiments only and is not intended to be limiting of present inventive concepts. Unless otherwise defined, all terms (including technical and scientific terms) used herein have the same meaning as commonly understood by one of ordinary skill in the art to which present inventive concepts belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

When an element is referred to as being “connected,” “coupled,” “responsive,” or variants thereof to another element, it can be directly connected, coupled, or responsive to the other element or intervening elements may be present. In contrast, when an element is referred to as being “directly connected,” “directly coupled,” “directly responsive,” or variants thereof to another element, there are no intervening elements present. Like numbers refer to like elements throughout. Furthermore, “coupled,” “connected,” “responsive,” or variants thereof as used herein may include wirelessly coupled, connected, or responsive. As used herein, the singular forms “a,” “an” and “the” are intended to include the plural forms as well, unless the context clearly indicates otherwise. Well-known functions or constructions may not be described in detail for brevity and/or clarity. The term “and/or” includes any and all combinations of one or more of the associated listed items.

It will be understood that although the terms first, second, third, etc. may be used herein to describe various elements/operations, these elements/operations should not be limited by these terms. These terms are only used to distinguish one element/operation from another element/operation. Thus, a first element/operation in some embodiments could be termed a second element/operation in other embodiments without departing from the teachings of present inventive concepts. The same reference numerals or the same reference designators denote the same or similar elements throughout the specification.

As used herein, the terms “comprise,” “comprising,” “comprises,” “include,” “including,” “includes,” “have,” “has,” “having,” or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.,” which derives from the Latin phrase “exempli gratia,” may be used to introduce or specify a general example or examples of a previously mentioned item, and is not intended to be limiting of such item. The common abbreviation “i.e.,” which derives from the Latin phrase “id est,” may be used to specify a particular item from a more general recitation.

Example embodiments are described herein with reference to block diagrams and/or flowchart illustrations of computer-implemented methods, apparatus (systems and/or devices) and/or computer program products. It is understood that a block of the block diagrams and/or flowchart illustrations, and combinations of blocks in the block diagrams and/or flowchart illustrations, can be implemented by computer program instructions that are performed by one or more computer circuits. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and/or flowchart block or blocks, and thereby create means (functionality) and/or structure for implementing the functions/acts specified in the block diagrams and/or flowchart block(s).

These computer program instructions may also be stored in a tangible computer-readable medium that can direct a computer or other programmable data processing apparatus to function in a particular manner, such that the instructions stored in the computer-readable medium produce an article of manufacture including instructions which implement the functions/acts specified in the block diagrams and/or flowchart block or blocks. Accordingly, embodiments of present inventive concepts may be embodied in hardware and/or in software (including firmware, resident software, micro-code, etc.) that runs on a processor such as a digital signal processor, which may collectively be referred to as “circuitry,” “a module” or variants thereof.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Finally, other blocks may be added/inserted between the blocks that are illustrated, and/or blocks/operations may be omitted without departing from the scope of inventive concepts. Moreover, although some of the diagrams include arrows on communication paths to show a primary direction of communication, it is to be understood that communication may occur in the opposite direction to the depicted arrows.

Many variations and modifications can be made to the embodiments without substantially departing from the principles of the present inventive concepts. All such variations and modifications are intended to be included herein within the scope of present inventive concepts. Accordingly, the above disclosed subject matter is to be considered illustrative, and not restrictive, and the appended examples of embodiments are intended to cover all such modifications, enhancements, and other embodiments, which fall within the spirit and scope of present inventive concepts. Thus, to the maximum extent allowed by law, the scope of present inventive concepts are to be determined by the broadest permissible interpretation of the present disclosure including the following examples of embodiments and their equivalents, and shall not be restricted or limited by the foregoing detailed description.

What is claimed is:

1. A method for preparing an acetabulum of a patient in a total hip arthroplasty (THA) procedure, comprising:
assembling a navigated reamer construct including a reamer basket;
providing a camera tracking system adapted to track a pose of the navigated reamer construct relative to the patient, wherein the camera tracking system is in communication with a computer platform;
inserting the reamer basket of the navigated reamer construct into the acetabulum;
displaying navigation guidance to a user, wherein the navigation guidance comprises one or more of a depth and a planned center of an acetabular shell;
reaming the acetabulum with the navigated reamer construct based on the navigation guidance displayed to the user, to remove damaged bone therefrom; and
removing the navigated reamer construct from acetabulum.

2. The method of claim 1, wherein assembling the navigated reamer construct comprises:
operatively coupling a power system to a reamer driver;
affixing the reamer basket of a selected size to a distal end of the reamer driver; and
inputting data indicating the selected size of the reamer basket into the computer platform.

3. The method of claim 2, wherein assembling the navigated reamer construct comprises:
affixing a tracking array to the reamer driver, wherein the tracking array comprises a plurality of reference elements adapted for tracking by the camera tracking system.

4. The method of claim 2, wherein the inputting further comprises using a touch screen or an extended reality (XR) interaction with an XR headset.

5. The method of claim 1, wherein the displaying further comprises displaying the navigation guidance to the user on one or more of a display screen or an extended reality (XR) headset in communication with the computer platform.

6. The method of claim 1, further comprising performing two or more iterations of any two or more processes selected from: the assembling, inserting, displaying, reaming, and removing processes.

7. The method of claim 6, wherein a first selected size of the reamer basket is used in a first iteration, and a second selected size of the reamer basket is used in a second iteration.

8. The method of claim 1, further comprising:
affixing a patient reference array to a rigid anatomical feature of the patient prior to the inserting, wherein the patient reference array comprises a plurality of reference elements adapted for tracking by the camera tracking system.

9. A method for placing an acetabular shell in a prepared acetabulum of a patient in a total hip arthroplasty (THA) procedure, comprising:
assembling a navigated inserter construct;
providing a camera tracking system adapted to track a pose of the navigated inserter construct relative to the patient, wherein the camera tracking system is in communication with a computer platform;
displaying navigation guidance to a user;
inserting the acetabular shell into the prepared acetabulum of the patient using the navigated inserter construct;
setting a trajectory of the navigated inserter construct based on the navigation guidance;
impacting the navigated inserter construct along the trajectory; and
removing the navigated inserter construct from the acetabular shell.

10. The method of claim 9, wherein assembling the navigated inserter construct comprises:
affixing a tracking array to an inserter, wherein the tracking array is adapted for tracking a pose of the navigated inserter construct by the camera tracking system.

11. The method of claim 9, wherein assembling the navigated inserter construct further comprises:
attaching the acetabular shell to the navigated inserter construct, wherein the acetabular shell is of a selected size.

12. The method of claim 9, wherein the trajectory is set based on one or more of: a target height, a target inclination angle, a target version angle, a target depth, or a screw location via rotation of the acetabular shell in relation to bone.

13. The method of claim 9, further comprising placing a liner in the acetabular shell after removing the navigated inserter construct therefrom.

14. The method of claim 13, further comprising:
prior to placing the liner, inserting a fixation structure into a fixation aperture in the acetabular shell, thereby augmenting fixation of the acetabular shell in the acetabulum.

15. The method of claim 9, further comprising:
removing one or both of a trial shell and a liner from the acetabulum prior to the inserting.

16. The method of claim 9, further comprising:
prior to inserting the navigated inserter construct into the acetabulum, registering a location of a fixation aperture of the acetabular shell to the computer platform.

17. The method of claim 9, further comprising:
affixing a patient reference array to the patient, wherein the patient reference array comprises a plurality of reference elements, and is adapted for tracking by the camera tracking system.

18. The method of claim 9, wherein the displaying further comprises displaying the navigation guidance to the user on one or more of a display screen or an extended reality (XR) headset in communication with the computer platform.

19. A method for performing a navigation-assisted total hip arthroplasty (THA) procedure, comprising:
providing a camera tracking system adapted to track one or more tracking arrays, and a computer platform in communication with the camera tracking system;
affixing a first tracking array to a pelvis of a patient, wherein the first tracking array comprises a plurality of reference elements adapted for tracking by the camera tracking system;
assembling a navigated reamer construct comprising a reamer, a reamer basket of a selected size, and a second tracking array, wherein the second tracking array comprises a plurality of reference elements adapted for tracking a pose of the navigated reamer construct by the camera tracking system;
inserting the reamer basket of the navigated reamer construct into an acetabulum of the patient;
displaying navigation guidance to a user for the navigated reamer construct;
reaming the acetabulum with the navigated reamer construct, based on the navigation guidance displayed to the user;
removing the navigated reamer construct from the acetabulum;
assembling a navigated inserter construct comprising an inserter, an acetabular shell coupled thereto, and a third tracking array comprising a plurality of reference elements adapted for tracking a pose of the navigated inserter construct by the camera tracking system;
displaying navigation guidance to a user for the navigated inserter construct;
inserting the acetabular shell into the acetabulum of the patient using the navigated inserter construct;
setting a trajectory of the navigated inserter construct based on the navigation guidance;
impacting the navigated inserter construct along the trajectory; and
removing the navigated inserter construct from the acetabular shell.

20. The method of claim 19, further comprising:
inputting data indicating the selected size of the reamer basket into the computer platform by using a touch screen or an extended reality (XR) interaction with an XR headset that is in communication with the computer platform,
wherein the displaying further comprises displaying the navigation guidance to the user on one or more of a display screen or the XR headset.
